(19) United States
(12) Patent Application Publication
(10) Pub. No.: US 2007/0219592 A1
(43) Pub. Date: Sep. 20, 2007

(54) Cardiac Pacemaker and/or ICD Control and Monitor

(75) Inventors: Noel Axelrod, Jerusalem (IL); Eran Ofek, Modi'in (IL); Douglas P. Zipes, Carmel, IN (US)

Correspondence Address: EDWARD S. SHERMAN, ESQ., 3554 ROUND BARN BLVD., SUITE 303, SANTA ROSA, CA, 95403, US

(73) Assignee: PHYSICAL LOGIC AG, Zug (CH)

(21) Appl. No.: 11/678,689

(22) Filed: Feb. 26, 2007

Related U.S. Application Data
(63) Continuation-in-part of application No. 11/674,951, filed on Feb. 14, 2007.
(60) Provisional application No. 60/777,648, filed on Feb. 28, 2006.

(51) Int. Cl. A61N 1/36

(52) U.S. Cl. 607/19; 607/5

(57) ABSTRACT

A cardiac pacemaker and/or ICD device deploys a plurality of three-dimensional accelerometers to characterize and distinguish between the local motion of the heart and the gross movement of the patient. The relative difference between these movements is used to distinguish between false negatives results of the electrogram reading to avoid triggering an unneeded defibrillation pulse, or increasing the pacing rate when the patient is exercising.

### CROSS REFERENCE TO RELATED APPLICATIONS

The present application is a continuation-in-part of the U.S. Non-provisional patent application for an “Accelerometer” filed on Feb. 14, 2007, and assigned application Ser. No. 11/674,951,which claims priority to the U.S. provisional patent application for an “Accelerometer” filed Feb. 21, 2006, and assigned application Ser. No. 60/775,530, both of which are incorporated herein by reference.

The present application also claims priority to the U.S. provisional patent application entitled “Cardiac Pacemaker and/or ICD Control and Monitor”, filed on Feb. 28, 2006, and assigned application Ser. No. 60/777,648, which is incorporated herein by reference.

### BACKGROUND OF INVENTION

The present invention relates to an apparatus and method for detecting and analyzing a patient's cardiac function to optimize the efficient and corrective operation of a pacemaker or ICD.

Pacemakers are implantable medical devices that replace or supplement a damaged or weak heart's ability to control the cardiac rhythm by periodic electric discharge, which initiates the contraction of the different portions of the cardiac muscle in a coordinated fashion for the efficient pumping of blood.

Implantable cardioverter defibrillators (ICD) are implantable medical devices that detect the lack of a regular cardiac rhythm and apply a large electric pulse that effectively shocks the heart from a disorganized and sporadic weak muscular contraction, known as fibrillation, back into the strong regular contraction necessary for supplying tissue with blood oxygenated by the lungs. The ICD can also terminate an abnormal fast rhythm by delivering competing low voltage electrical pulses called antitachycardia pacing (ATP). In virtually all modern ICD's both the capability for pacing and defibrillation are present. Such devices deploy a plurality of electrical leads into different chambers and portions of the heart both to monitor cardiac function, through an electrogram (EG), apply a low voltage pacing pulse to the heart, and when determined to be therapeutically essential apply a high voltage pulse for defibrillation of the heart.

Some pacemakers have the limitation that they are set at a rate and power level that remains constant while implanted in the patient. Unlike the natural physiological pacing function of the heart, they cannot set a faster rate of pumping when the patient is exerting more energy and needs a greater supply of oxygenated blood to satisfy the metabolic demands of active muscle tissue. Other pacemakers are rate responsive and can increase rate by monitoring a number of physiologic functions such as tracking body movement, respiration, QT interval, and other end points.

ICD's, while responsible for saving and prolonging the lives of thousands of patients, also have the undesirable potential for applying painful shocks that are either unnecessary, due to a false positive reading that the patient was in fibrillation from the internal EG, or are delivered when the patient is still conscious, to cardiovert or defibrillate the heart and restore normal cardiac rhythm.

It is therefore a first object of the present invention to provide an improved method of regulating the discharge or electrical pacing rate of an implantable or other cardiac pacemaker that is responsive to the patient's level of physical activity and/or blood oxygen demand.

It is yet another objective of the invention to provide a method of verifying the results of the electrogram measurement of the ICD device to avoid false measurements and unnecessary shocks.

It is yet another objective of the invention to provide improved arrhythmia detection and recognition that supplements the results of an electrogram.

It is a further object of the current invention to regulate an ICD device to provide a more appropriate discharge, and thus more proportionately treat a defibrillation or related cardiac condition. Achieving this objective not only avoid shocks that are stronger, but also conserves device energy and battery resources, thus prolonging the lifetime of the ICD.

### SUMMARY OF INVENTION

In the present invention, the first object of providing variable cardiac pacing to accommodate patient activity level is achieved by providing a 1st 3-D accelerometer (3DA) coupled to the heart and 2nd 3DA not coupled to the heart along with means for detecting the comparative movement between 1st and 2nd accelerometer.

Another object of the invention is achieved by also providing an improved means to increase the pacing frequency in response to the patient's differential reading between the first and second 3DA, which indicates the level of physical activity.

Another object of the invention of improving the reliability of the ICD is achieved by providing an accelerometer and/or other motion sensors coupled to the heart along with means for detecting the comparative movement between the 1st and 2nd accelerometer, as well as a means to verify the accuracy of electrogram measurements and confirm that fibrillation is occurring by the lack or nature of the movement or vibration associated with the heart wall.

Another aspect of providing a proportionally appropriate therapeutic discharge from an ICD is achieved by providing a plurality of accelerometer and/or other motion sensors coupled to the heart with computational means to quantify at least one of the location and magnitude of localized cardiac fibrillation and/or fluttering, as well as means to regulate the magnitude and location or the electric discharge in response thereto.

The above and other objects, effects, features, and advantages of the present invention will become more apparent from the following description of the embodiments thereof taken in conjunction with the accompanying drawings.

### BRIEF DESCRIPTION OF DRAWINGS

FIG. 1 is a schematic illustration of a patient being diagnosed with the inventive apparatus.

FIG. 2 is a flow chart illustrating the data collection and analysis of the signals obtained from the sensors shown in FIG. 1.

FIG. 3 is schematic illustration of the interior a patient's heart showing the potential location of alternative sensors as integrated with pacing or ICD leads and devices.

FIG. 4 is schematic illustration of a partial interior of a patient's heart showing the potential location of alternative sensors as integrated with multiple ICD electrodes.

### DETAILED DESCRIPTION

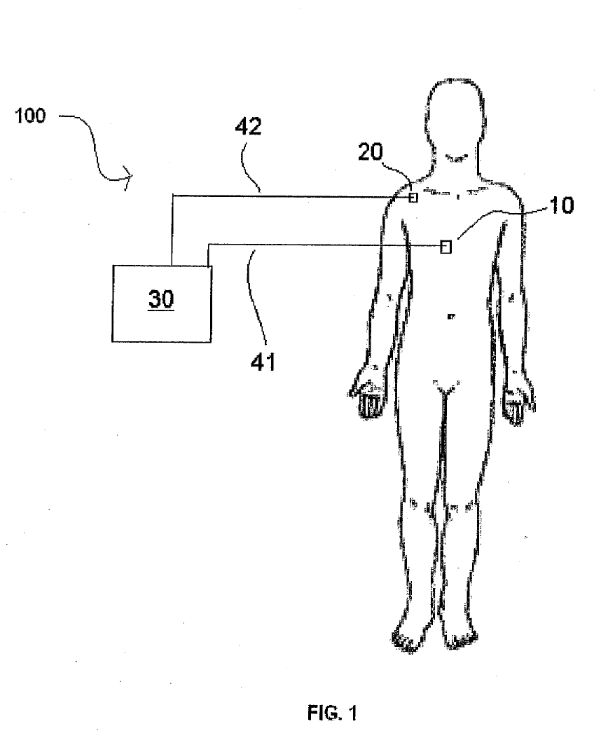

Referring to FIGS. 1 through 4, wherein like reference numerals refer to like components in the various views, there is illustrated therein a new and improved method and apparatus for the cardiac pacing and monitoring, as well as control of implantable cardiac defibrillators, generally denominated 100 herein.

First, it should be appreciated that the heart is a complex organ both physiologically and structurally. On a structure level, the heart is a two-stage pump with four chambers and two pairs of valves. The inventors have realized that the heart's physiological characteristics may be diagnosed, in addition to traditional methods, by observing the structural variation of the heart during the cardiac cycle. Unlike most mechanical pumps, the heart itself undergoes changes in its external shape due to both the contraction of the cardiac muscle as well as the flow of blood into and out of the chambers.

Detection of body micro-vibration is known in the art, see for example R. Strum, R, B. Nigg and E. A. Koller, “The impact of cardiac activity on triaxially recorded endogenous micro-vibrations of the body”, European Journal of Applied Physiology, vol. 44, pp. 83-96, 1980. Strum et al. evaluated the relationship between the cardiac activity and the micro-vibrations of the body and concluded that the most important source of whole-body micro vibrations is the cardiac activity.

Further, in U.S. Pat. No. 6,328,698 (to Matsumoto and issued Dec. 11, 2001), which is incorporated herein by reference, there is disclosed a diagnostic system and method for coronary artery disease which is operative to detect vibration signal of murmur deriving from stenosis of coronary artery in its early stages. The vibration signals are detected using one or a plurality of laser source head and vibration detective sensors with laser displacement gage and three-axial accelerometer, and the detector of vibration signal of environmental noise has three-axial accelerometer and supersensitive microphone.

While some of these methods can very accurately detect the presence of congenital or degenerative disorders very effectively, they generally require varying degrees of either complex equipment and/or expert human analysis, that may prevent the patient from undergoing normal activity when the results are the most clinically meaningful.

The inventors have further appreciated that due to the inherent electro-mechanical coupling of cardiac function, as well as the elastic nature of both muscle and vascular tissue, the pumping action will generate numerous vibrations that propagate in multiple directions.

The inventors have come to realize and appreciate that distinguishing the 3-D movement of the heart muscle and tissue from acceleration and/or vibration measurements, when coupled and compared with 3-D movement of the patient's body, can provide feedback to a pacemaker or ICD that is therapeutically useful.

The inventors have further realized and appreciated that such vibration can be analyzed to detect and quantify the state of cardiac health, and in particular to verify normal heart function as well as detecting the location and magnitude of abnormal conditions, and in particular cardiac fibrillation.

In accordance with one aspect of the present invention, movements of the cardiac wall and associated vibrations are detected and analyzed for use in the operation and control of cardiac pacemaker and ICD. In such methods, as shown in FIG. 1, the patient 1 has at least two sensors, 10 and 20, placed either on the skin, but preferably with at least the primary sensor 10 in close proximity to the heart, and more preferably on a catheter that is anchored to the heart muscle wall. A secondary sensor 20 is placed more distal from the heart. The second sensor is intended, in some embodiments, to detect movement and/or vibration arising from other than pure physiological functions, such as the gross motion or movement of the patient, or from an external source such as floor vibrations. The primary sensor 10 is placed closer to the heart to detect either vibrations arising from the movement of the heart and the blood being pumped therein in the cardiac cycle and/or movement of the heart wall. The signals from the sensors are received by a processing unit 30. In some embodiments, each sensor is preferably an accelerometer capable of measuring independent acceleration in three orthogonal directions. When the output of the first or primary sensor is suitably filtered to remove noise and vibrations not associated with the cardiac cycle, the amplitude of the remaining acceleration and vibrations represents the movement of the heart in the three directions. Such filtering, and other computations, are performed by the processing unit 30. While it is anticipated that certain types of sensors, can be used when the patient is in a supine position and connected to the processing unit 30 by cables 41 and 42, it is preferable that they are worn during exercise or normal use. Accordingly, it is more preferable that the processing unit 30 and cables 41 and 42 are internal to the patient, as well as sensors 10 and 20, such as in or a part of the pacemaker or ICD. In addition, the processing unit 30 is preferably integrated into at least one of leads, stent, or device/battery package.

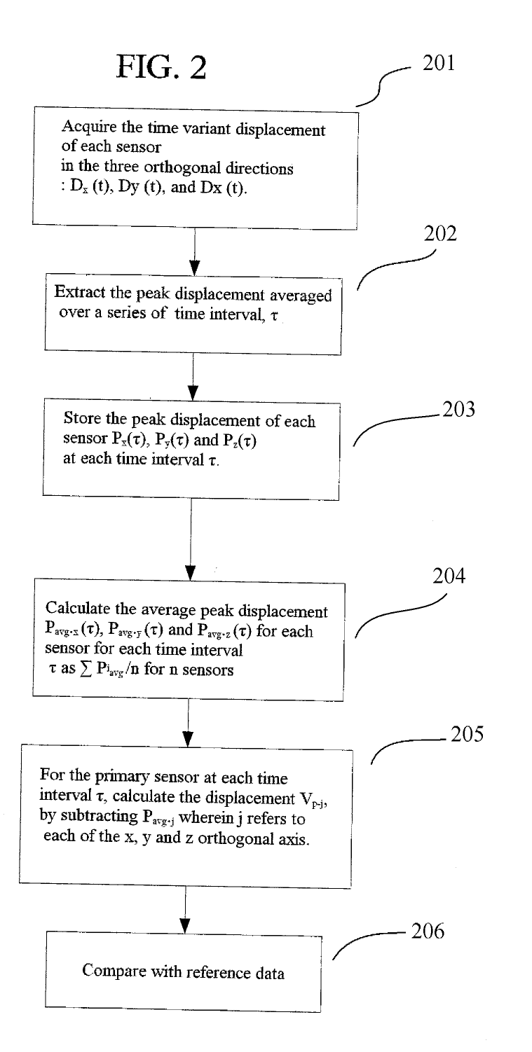

In deploying the sensors 10 and 20 as a mechanical heart motion monitor it is preferable to use a plurality of sensors that surround the heart to more accurately separate and filter acceleration and/or vibrations not associated with the heart's motion. The output of each sensor can be compared with the average output of every other sensor, wherein the average output is filtered out as background noise. In this manner, vibrations arising from the more remote sensors not associated with the heart's motion will be removed. The basic analysis algorithm is further explained with reference to FIG. 2.

As shown in the flow chart of FIG. 2, in the first step in the process 201 the sensors acquire the time variant displacement of each sensor in the three orthogonal directions: Dₓ (t), D_{y} (t), and D_{z} (t). In the next step in the process, 202, the peak displacement of the vibration sensor, that is the amplitude of the vibration, is extracted as the average over a series of time intervals; τ. preferably, the cardiac cycle is divided into a sufficient number of time intervals to fully resolve each critical operative stage of the cardiac activity. In the next step in the process, 203, the peak displacements of each sensor Pₓ (τ), P_{y} (τ) and P_{z} (τ) at each time interval τ are stored for further calculation. However, such storage can be merely transitory for a very brief time period for continuous calculation in step 204. In step 204, the average peak displacement P_{avg·x} (τ), P_{avg·y} (τ) and P_{avg·z} (τ) for each sensor for each time interval τ is calculated as ΣPⁱ_{avg}/n for n sensors. In the next step in the process, 205, for the primary sensor at each time interval τ, the displacement Vₚ₋ⱼ, is calculated by subtracting P_{avg·j} wherein j refers to each of the x, y and z orthogonal axis. Other aspects of the process, step 206, include comparing resultant process signals to a patient or generic reference signal or a pattern characteristic of the normal hearts movement, and extracting the heart rate from the periodic changes in any of the acquired or derived signal. Preferably, the sensors 10 and/or 20 include integrated microelectronics in the sensor, for analog and digital processing of 3D cardiac wall motion for improved arrhythmia detection and recognition.

Further, as it is anticipated that via electromechanical coupling, the physical movement of heart muscle mass will correlate with the electrical activity associated with one or more of the PQRS and T waves of ECG. U.S. Pat. No. 5,554,177 (to Kieval for a “Method and apparatus to optimize pacing based on the intensity of acoustic signal” and issued Sep. 10, 1996), which is incorporated herein by reference, illustrates the general correlation of gross audio frequency vibrations with the electrical activity recorded by ECG, as well as other cardiac activity detectable by Doppler methods. Thus, it is expected that the coupled analysis of the three-dimensional digital cardiac wall motion with the cardiac electrical activity can be used to detect various cardiovascular problems; examples of such problems can include various cardiac arrhythmias, irregularities in blood flow to the heart etc. Other expected benefits of deploying the above components for such three dimensional digital cardiac wall motion analysis include, without limitation cardiac tissue segment fibrillation and fluttering analysis (such as the location and type), heart beat contractility analysis (i.e. motion amplitude) and rhythm detection and interpretation.

Accordingly, when the above elements of the invention are deployed in an ICD, analysis and comparison of the movement of the heart wall can be used to prevent unnecessary shock delivery by providing the ICD with an exact heart rate derived from mechanical analysis. Thus, another embodiment of the current invention is to provide accurate rate detection without electrical sensing, used to confirm electrical rate detection. In other embodiments, the ICD can be programmed to override potentially false sensing electrical sensing, and thus prevent or reduce the potential for inappropriate therapy

Thus, another aspect of the invention is the method of deploying device 100 in communication with an ICD, comparing the movement to generate a 3-D profile of the heart movement, determining if the heart is in fibrillation from the electrograms, verifying fibrillation by comparison of the instant 3-D profile with the expected heart movement in a reference profile and then applying a defibrillating electric discharge if fibrillation is verified

Further, the comparison of the expended mechanical movement signature of the heart can be used to distinguish normal rapid (SVT) or a hemodynamically tolerated ventricular tachycardia, from abnormal rapid (VT/VF) contractions such that either an ICD or pacemaker provides more accurate treatment application.

It is also anticipated that device and disclosed methods can locate presence and site of wall motion abnormalities that may occur during ischemia. For example when the electrical discharge of the ICD is coupled with the processing and analysis acquired from hemodynamic sensing (i.e. at least one or more of blood pressure, flow velocity and blood chemistry) shock delivery can be delayed until it is hemodynamically required, thus permitting longer anti-tachycardia pacing (ATP) attempts.

In some applications it is desirable that each of the primary and secondary sensors or a plurality of primary sensors is sufficiently small so they can be worn indefinitely on the patient's skin, to optionally provide continuous measurement. U.S. Pat. No. 6,118,208 (which issued to Green, et al., Sep. 12, 2000 and is incorporated herein by reference) discloses an acoustic or vibration sensor particularly useful in detecting nano-vibrations. Thus, other suitable sensors include, without limitation, accelerometers, hydrophones, microphones, laser velocimeters, strain gages, and motion detectors. Such alternative types and locations for sensors 10 and 20 are may also be deployed as part of device 100 as part of or in signal communication with a subcutanouos ICD, utilizing the mechanical movement signature to avoid muscle noise and need for other biologic sensors. A preferred types of accelerometer sensor is disclosed in the parent and commonly owned U.S. non-provisional patent application having Ser. No. 11/674,951, which is incorporated herein by reference, as it is highly sensitive at a small size and consumes a relatively small amount of power.

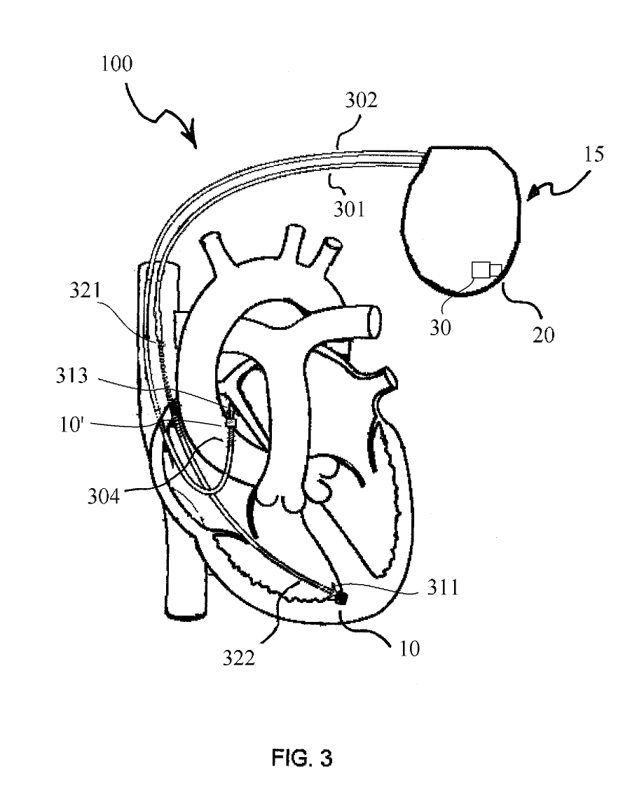

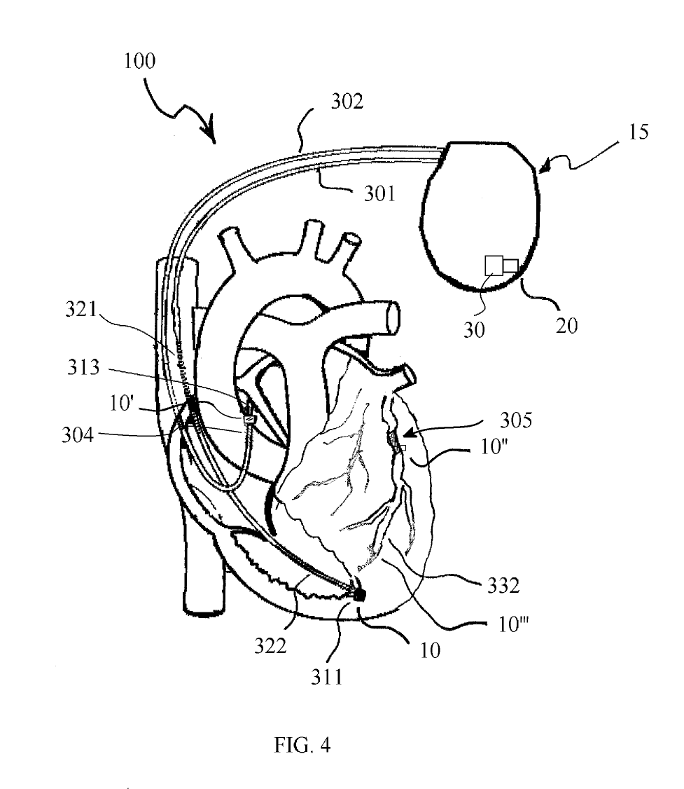

The use of multiple primary sensors, distributed as shown in FIG. 3 or FIG. 4 also provides a means to locate the presence and site of wall motion fibrillation and fluttering. Preferably, the primary sensors are embedded in either pacing leads, ICD electrodes or stents. As shown in FIG. 3, a first catheter type lead 301 is positioned or deposed in the right ventricle being attached to the heart wall by anchor 311 at its distal end. The catheter/lead has a first electrical terminal or electrode 321 positioned or deposed in the right atrium with the second terminal 322 positioned or deposed just above anchor 311 and the wall of the right ventricle. A first primary sensor 10 is mechanically coupled to the heart wall by anchor 311. The proximal end of lead 301 is connected to ICD device 15, which now includes secondary sensor 30. Generally, the ICD device 15 applies a therapeutic shock between terminals 311 and 322.

A second catheter type lead 302 terminates with anchor 313 connecting the distal end of this lead to the heart wall in the right atrium so that the corresponding terminal 304 is in electrical communication the atrium. The second catheter type lead 302 has a second primary sensor 10′ just above anchor 313. Alternatively, each or a single catheter type may deploy to or more primary sensors. Generally, terminal 304 is used for pacing, if required. However, the pair of electrical terminals is potentially available for recording an electrogram to determine the health of the patient (such as by heart rate variability) or to detect cardiac arrhythmias and/or provide feedback between the desired pacing frequency and the actual heart rate as measured from the electrograms. To the extent that any of the electrogram sensing leads fail or give false positive readings of arrhythmias, such as due to the patient's movement, the output of one or more primary sensors can be used to confirm such readings. One useful aspect of this embodiment of the invention is the prevention of unnecessary shock or ATP delivery.

FIG. 4 illustrates an alternative embodiment of the invention, which includes the device 15 and leads 301 and 302 of FIG. 3. However, only the right side of the heart is shown in section, with the surface of the heart showing the right coronary artery having a stent disposed 303 disposed therein. This stent 305 may include one or more additional primary sensors 10″ and 10′″, all or which are in signal communication with ICD/pacemaker 15 and associated processing unit 30. It should be understood in this case, that it is preferable that the signal communication with processing unit 30 is wireless. h.)

Processing unit 30, being in communication with multiple primary sensors 10 is able to calculate from at least one of local heart wall accelerations and or nano-vibrations to ascertain the focus of an electrical abnormality, such as fibrillation from the characteristic flutter and vibration. Thus, once the location of the local abnormality is identified, the processing unit 30 is operative to calculate which of the pairs of electrodes associated with catheters 301, 302 and 303 is best positioned to apply a voltage at a location that will correct the abnormality. It is anticipated that the ability to apply a localized voltage to localized fibrillation before it has spread to include the entire heart will improve shocking process efficiency for device and patient benefit (i.e., lower energy delivery per shock that can be directed at specific targets). It should be appreciated that although some aspects of the invention require one or more of sensors 10 and 20 to be a 3-dimensional accelerometer, it should be understood that other types of mechanical sensors, and in particular nano-vibration sensors may be preferable as a means to locate the source of fibrillation or other abnormal condition in the heart. In addition other types of transducers that measure for example blood chemistry, tissue ischemia, blood pressure and/or flow and the like may be used in combination or in place of sensors 10 to determine the focus of a cardiac abnormality and apply a similar localized therapeutic electrical shock

U.S. Pat. No. 5,617,869 to Austin, et al., which is incorporated herein by reference, issued on Apr. 8, 1997 and discloses a method and apparatus for locating artery stenosis in blood vessels utilizing multiple sensors. As the localization of the artery stenosis having a characteristic noise or vibration can be achieved through array signal processing it should be apparent to one of ordinary skill in this art to deploy sensors 10 and 20 in a similar manner to locate either stenosis in the coronary arteries and/or the location of fibrillation.

Another aspect of the invention is the deployment of the primary and secondary sensor as integrated or in signal communication with a pacemaker. In this embodiment, the 3D movement can be analyzed to determine if the patient is engaging in physical activity. For example, a patient jogging would have a periodic variation in the vertical acceleration in proportion to their stride and speed, which could be measured by the secondary sensor without reference to the primary sensor, but is preferably measured and confirmed by a plurality of sensors in different locations to distinguish between artifacts Thus, the pacemaker frequency could then be modulating to provide a faster “pulse” appropriate to the patients level of physical exertion.

While the invention has been described in connection with a preferred embodiment, it is not intended to limit the scope of the invention to the particular form set forth, but on the contrary, it is intended to cover such alternatives, modifications, and equivalents as may be within the spirit and scope of the invention as defined by the appended claims.

1. A biomedical transducer comprising:
a) a first 3DA to be disposed on the subject to measure the gross motion thereof,
b) a second 3DA to be disposed in close proximity to the subject's heart to measure the local movement of the heart,
c) means for comparing the output of the first and second 3DA's to determine the relative movement of the heart,
d) means to modulate the output of a pacemaker or ICD device in response to the relative movement of at least one of the heart and the subject.

2. A biomedical transducer according to claim 1 and further comprising:
a) means to detect and locate the source of fibrillation in the heart,
b) means to apply a defibrillating or other therapeutic electric discharge in close proximity to the source of fibrillation or fluttering in the heart.

3. A biomedical transducer according to claim 1 and further comprising means to modulate the power of a defibrillating or other therapeutic electric discharge.

4. A biomedical transducer according to claim 2 and further comprising means to modulate the power of the defibrillating or other therapeutic electric discharge.

5. A method of treating cardiac patients, the method comprising the steps of:
a) providing the patient with at least one of a cardiac pacemaker or ICD,
b) providing at least a first 3DA disposed on the patient to measure the gross motion thereof,
c) providing at least a second 3DA disposed in close proximity to the heart of the patient to measure the local movement of the heart,
d) determining the patient's level of physical activity from at least one of the out of the first and second 3DA,
e) modulating the output of the pacemaker in response to patient's level of physical activity.

6. A method according to claim 5 wherein the pacemaker or ICD is implanted within the patient.

7. A method according to claim 5 wherein the first 3DA is in the portion of the pacemaker or ICD containing the battery and the second 3DA is on an electrical lead implanted in or in electrical communication with the heart.

8. A method of treating cardiac patients, the method comprising the steps of
a) providing the patient with an ICD,
b) providing at least a first 3DA disposed on the patient to measure the gross motion thereof,
c) providing at least a second 3DA disposed in close proximity to the heart of the patient,
d) comparing the movement between 1st and 2nd accelerometer to measure the local movement of the heart,
e) verifying the accuracy of electrogram measurements by the ICD by confirming that the movement of a least a portion of the heart corresponds with electrical activity detected by the electrogram,
f) applying at least one appropriate therapeutic shock via the ICD when the electrogram measurement is verified.

9. A method according to claim 8 wherein said step of detecting computing determining is in response to the ICD detecting at least one of defibrillation and tachycardia.

10. A method according to claim 8 wherein the first 3DA is in the portion of the pacemaker or ICD containing the battery and the second 3DA is on an electrical lead implanted in or in electrical communication with the heart.

11. A method according to claim 8 wherein the pacemaker or ICD is implanted within the patient.

12. A method of treating cardiac patients, the method comprising the steps of:
a) providing the patient with an ICD,
b) providing at least a first 3DA disposed on the patient to measure the gross motion thereof,
c) providing at least one additional accelerometer disposed in close proximity to a local region of the heart of the patient,
d) comparing the output of the first 3DA and the least one additional accelerometer to determine the relative local movement of at least a portion of the heart,
e) computing at least one of the location and magnitude of cardiac fibrillation and/or fluttering from said comparison step,
f) determining the appropriate therapeutic discharge from the ICD for least one of the location and magnitude of the cardiac fibrillation and/or fluttering form the said comparison.

13. A method according to claim 12 wherein the first 3DA is in the portion of the pacemaker or ICD containing the battery and the second accelerometer is on an electrical lead implanted in or in electrical communication with the heart.

14. A method according to claim 12 wherein said step of detecting computing determining is in response to the ICD detecting at least one of defibrillation and tachycardia.

15. A method according to claim 12 wherein a plurality of additional accelerometers are disposed in close proximity to different local regions of the heart of the patient.

16. A method according to claim 12 wherein the second accelerometer is a two-dimensional accelerometer.

17. A method according to claim 12 wherein the second accelerometer is a 3DA.

18. A method according to claim 12 pacemaker or ICD is internal/external.

19. A method according to claim 17 wherein the first 3DA is in the portion of the ICD containing the battery and the second 3DA is on an electrical lead implanted in or in electrical communication with the heart.

20. A method according to claim 12 wherein said step of comparing the output of the first 3DA and the least one additional accelerometer to determine the relative local movement of at least a portion of the heart is in response to the ICD detecting at least one of defibrillation and tachycardia.

21. A method according to claim 12 wherein at least one accelerometer is coupled to a cardiac stent.
